(19) United States
(12) Patent Application Publication
(10) Pub. No.: US 2006/0265766 A1
(43) Pub. Date: Nov. 23, 2006

(54) Fish and the production thereof

(75) Inventors: David J. Kyle, Cantonsville, MD (US); Moti Harel, Baltimore, MD (US); John Piechocki, Odenton, MD (US)

Correspondence Address: DUANE MORRIS, LLP;IP DEPARTMENT, 30 SOUTH 17TH STREET, PHILADELPHIA, PA, 19103-4196, US

(73) Assignee: Advanced Bionutrition Corporation, Columbia, MD (US)

(21) Appl. No.: 10/549,730

(22) PCT Filed: Mar. 19, 2004
(86) PCT No.: PCT/US04/08561

§ 371 Date: Jul. 5, 2006

Related U.S. Application Data
(60) Provisional application No. 60/455,556, filed on Mar. 19, 2003.

(51) Int. Cl. A23L 1/325; A01K 67/033; A01K 67/027

(52) U.S. Cl. 800/13; 800/20; 426/643

(57) ABSTRACT

This invention is directed to the composition, method of production, and use of improved edible fish, crustacean, or mollusk products enriched with one or more components that provide health benefits to humans or other animals consuming the product.

### BACKGROUND OF THE INVENTION

Consumers have expressed a growing preference for “Organic” foods, which are certified by Government Agencies to contain no chemical substances, such as pesticides, hormones, or synthetic chemicals not normally found in the natural food. Consumer concerns about the appropriateness of wild-caught seafood for human consumption (due to environmental damage and over exploitation of natural resources), as well as the perceived inferiority of animals raised on diets containing ingredients derived from pollution-contaminated and/or declining wild resources (e.g., fishmeal and fish oil), have resulted in a need to develop an Organic aquaculture technology. Organic certification in the U.S. requires that the feeds provided to aquacultured species meet the requirements of the U.S. Organic Foods Protection Act of 1991 (OFPA) and the USDA National Organic Standards (USDA NOS, 2002), which states that the total ration be composed of products that are Organically produced, and if applicable, Organically handled. To our knowledge, there does not appear to be a diet for any aquatic animal on the market that can meet the requirements for 100% Organic certification as defined by USDA NOS. Particularly problematic is the requirement for fishmeal and/or fish oil in the feed for optimal growth of certain animals (Sargent and Tacon 1999), especially carnivorous species. The aquatic animals presently marketed as food are either wild catch or produced in semi-intensive or highly intensive production systems that would not qualify for Organic certification.

Aquatic animals, such as fish, mollusks, and crustaceans, generally contain more omega-3 long chain polyunsaturated fatty acids (LC-PUFAs) than terrestrial animals (Crawford, Bloom et al. 1999). However, these aquatic animals cannot synthesize sufficient omega-3 LC-PUFAs de novo, rather the omega-3 LC-PUFAs are obtained through the aquatic food web from the microalgae, phytoplankton, and zooplankton in the aquatic ecosystem (Kyle and Arterburn 1998). It is important that fish obtain dietary omega-3 LC-PUFAs for optimal growth and development. Wild species of marine fish, such as salmon and tuna, have relatively high levels of certain omega-3 LC-PUFAs, such as eicosapentaenoic acid (EPA) and docosahexaenoic acid (DHA), whereas warm freshwater fish, such as catfish and tilapia, have much lower levels of EPA and DHA (Chetty, Reavis et al. 1989).

The commercial husbandry of aquatic animals required development of diets, which provide optimal growth of fish, crustaceans or mollusks (Shephard and Bromage 1992; McVey 1993). Most commercial diets include fish byproducts (fishmeal and/or fish oil) as sources of easily digestible protein and omega-3 LC-PUFAs in sufficient quantity to provide optimal growth of the aquatic animal. Levels of DHA and EPA added to aquatic animal diets may vary from species to species as some species (e.g., seabream, halibut, cod, tuna) have a greater requirement for DHA and EPA than do other aquatic species (e.g., catfish, tilapia, mussels). Species requiring high amounts of DHA tend to be carnivorous fish, while herbivorous fish generally require lower amounts of DHA (Chetty, Reavis et al. 1989).

LC-PUFAs, such as DHA, have a significant health benefit to growing infants, nursing mothers, children and adults in general (Gormley 1999). Therefore, increased consumption of DHA is recommended to compensate for the omega-3 deficiency in the modern diet. Terrestrial plant and animal based foods are relatively deficient in omega-3 LC-PUFAs, particularly DHA and EPA, while containing high levels of omega-6 LC-PUFAs. Thus, the main source of DHA and EPA in modern human diets is aquatic animals, such as fish, crustaceans, and mollusks. The principal aquacultured fish species in the United States is catfish, one of the poorest sources of DHA and EPA of all fish species (USDA 2002). Thus, it would be of great health benefit to provide a source of catfish that would deliver DHA in levels similar to that of salmon or tuna. Prior to this invention, such high-DHA catfish or tilapia products have never before been obtained.

Although the diets of many farmed aquatic animal species are rich in the omega-6 fatty acid linoleic acid (LA), the conversion of LA to arachidonic acid (ARA) is relatively inefficient in many species and the level of ARA in the meat or oil fractions from these fish is quite low (USDA 2002). This is particularly true for aquatic species fed very high levels of fishmeal and fish oil. ARA may benefit the growth and development of humans and other animals, however sources of dietary ARA are limited to meat and eggs (Kyle 1997). For example, humans with liver disease are unable to convert LA to ARA, resulting in low circulating ARA levels (Pita, Rubio et al. 1997). This results in low levels of thromboxin A2 and prostaglandin E2, leading to excessive bleeding (Burke, Ling et al. 2001). Dietary ARA is an essential dietary component in cats (Salem and Pawlosky 1994). Thus, it would be advantageous to enrich the lipid fraction of the aquatic animal with ARA to provide a healthier dietary component for consumption by humans and other animals that may need to enrich their levels of ARA.

Carotenoids (e.g., astaxanthin) are responsible for the desirable body coloration in certain aquatic animals, such as shrimp and salmonid fish (Meyers and Latscha 1997). Appearance, both before and after cooking, is an important factor influencing the consumers' purchase of seafood products. Such carotenoids can be of direct dietary origin or derived from metabolic transformation of another dietary carotenoid (Meyers and Latscha 1997). Astaxanthin is the principal carotenoid in most aquatic animals and is added to many feeds (Meyers and Latscha 1997). Astaxanthin is obtained by wild aquatic animals from phytoplankton (algal) or zooplankton (copepods) sources, which are either directly or indirectly in the diet through the food web. Other xanthophylls (such as lutein or zeaxanthin) and other carotenes (such as lycopene or γ-carotene) are typically not found in aquatic animals to any great extent. In humans, however, lutein and zeaxanthin are associated with better eye health (Snodderly 1995). The occurrence of age related macular degeneration (AMD) has been inversely correlated with dietary intake of lutein and/or zeaxanthin (Rapp, Maple et al. 2000). Thus, the substitution of the astaxanthin in an edible aquatic animal with lutein and/or zeaxanthin or an enrichment containing lutein and/or zeaxanthin would provide a food product that carries an additional eye health benefit to the consumer.

Taurine is a sulfur-containing amino acid shown to have many physiological actions in humans (Lourenco and Camilo 2002). The capacity of humans to synthesize taurine is limited and conditional deficiencies can occur particularly in newborn infants (Chesney, Helms et al. 1998). Certain other mammals, such as cats, do not have the ability to synthesize taurine; taurine deficiencies in cats have been shown to lead to visual disorders (Neuringer, Imaki et al. 1987). Taurine is typically found in meat and fish products, but is not found in plants. Consequently, vegetarians who do not consume dairy or egg products may also exhibit taurine deficiencies. Taurine is found in the central nervous system, skeletal muscles and in the heart (Suleiman, Moffatt et al. 1997). It also appears to be involved in transport of potassium, sodium, calcium and magnesium in and out of cells (Tricarico, Barbieri et al. 2001). Taurine has been used as an inhibitory neurotransmitter in the treatment of epileptic seizures or other excitable brain states (Lombardini 1992) and may be beneficial in problems with retinal disease, depression, male infertility and cardiovascular disease (Lourenco and Camilo 2002). The inventors have recognized that taurine appears to be involved in almost every function in which DHA is required; in clinical conditions where there is a DHA deficiency there is also a taurine deficiency. Co-supplementation of DHA and taurine could therefore be a benefit in the treatment of many of these diseases including, but not limited to, diseases of the central nervous system (e.g., depression, attention deficit disorder (ADD), Alzheimer's Disease (AD), epilepsy, schizophrenia, bipolar disorder, etc.), cardiovascular disease (e.g., hypertension, dyslipidemia, angina, arrhythmia, etc.), or other metabolic diseases (e.g., diabetes, cystic fibrosis, muscular dystrophy, etc.). Furthermore, co-supplementation of DHA and taurine may be particularly beneficial in some nonhuman vertebrates such as, but not limited to, mammals (e.g., felines, canines, bovines, porcines, etc.), birds (e.g., chickens, turkeys, etc.), and fish (e.g., Ostiechthyean and Chondrichthyean species), as well as certain marine invertebrates, such as, but not limited to, mollusks and crustaceans. Taurine has been shown, for example, to be an essential dietary component for cats, therefore consumption of a fish product enriched in taurine would be particularly beneficial to cats.

Custom designed aquatic animal feeds and production methods that enable the production of fish (e.g., catfish, tilapia, etc.), mollusks (e.g., oyster, mussels, etc.) and crustaceans (e.g., lobster, shrimp, crab, etc.) that have improved LC-PUFA profiles to enhance consumer and animal health (e.g., elevate levels of DHA), or a distinct, pleasing visual profile rich in lutein and/or zeaxanthin, or an elevated level of the amino acid taurine, are novel and deliver a significant improvement over existing commercially available seafood products. As the wild catch decreases, such designer fish, shellfish and crustaceans will fill an increasing market demand while improving on the nutritional value delivered over existing wild caught and farmed animals.

### SUMMARY OF THE INVENTION

This invention is directed to the production and use of edible aquatic animals (e.g., fish, mollusks and crustaceans) that are highly enriched with one or more compounds that are of health benefit to humans or animals consuming said seafood. In particular, these aquatic animals can be enriched in certain LC-PUFAs (such as, but not limited to, DHA EPA, or ARA), certain amino acids (such as, but not limited to, taurine, arginine, or methionine), and certain carotenoids (such as, but not limited to, lutein, astaxanthin, canthaxanthin, zeaxanthin or lycopene). Methods are also contemplated to enable Organic certification of the aquacultured seafood product by replacement of fishmeal and/or fish oil components with defined components such as, but not limited to, microalgal biomass.

The problem to be solved is the cultivation of an aquatic animal with an enhanced nutritional value with a totally vegetarian diet. Although fishmeal can be replaced with certain plant-derived protein sources, such as corn meal or soy meal, fish oil must still be used for optimal growth of the animal (i.e., to provide DHA, EPA, or ARA), thereby rendering such feeds as non-vegetarian and not certifiable under current “Organic” labeling. The inventors have solved the problem through the use of certain vegetarian sources of DHA, EPA and ARA.

A second problem to be solved is the provision of an aquatic animal product that will deliver a therapeutic dose of lutein and/or zeaxanthin that could be used to supplement the diet of individuals or animals with visual disorders, such as, but not limited to, macular degeneration, retinitis pigmentosa, and cataracts. The inventors have solved this problem by the provision of certain vegetarian sources of dietary lutein, zeaxanthin or lycopene such as, but not limited to, marigold petals, Lycium Chinese Mill berries, tomato processing waste, certain bacteria (e.g., Flavobacterium) and/or certain microalgae (e.g., Chlorella, Dunaliella, Nannochloropsis). The inventors have also discovered that the co-supplementation of the carotenoid with phospholipids, such as, but not limited to, soy lecithin, egg lecithin, or DHA-rich phospholipid extracts from algae, results in an unexpected increased bioavailability of carotenoid pigment.

A third problem to be solved is to increase the EPA, DHA, ARA and alpha linolenic acid (ALA) content of aquatic species, which typically have a very low level of these fatty acids (e.g., catfish, tilapia, and shrimp), and still maintain a vegetarian feed; thereby allowing “Organic” certification of the final product. The inventors have solved the problem by supplementing the feed of the aquatic animal with a microalgal or fungal feed in such a way that the DHA content of the aquatic animal is at least 50% greater than that of the wild caught animal or 100% greater than that of the current aquacultured animal.

A fourth problem to be solved is to increase the level of the amino acid taurine in an aquatic species of fish, mollusk or crustacean. This is particularly problematic while maintaining an Organic source and thereby allowing an “Organic” certification to the final product. The inventors have solved the problem by supplementing the feed of the aquatic animal with a microbial source of taurine in the feed in such a way that the taurine content of the aquatic animal is at least 50% higher that that of the current aquacultured animal.

### DETAILED DESCRIPTION AND EMBODIMENTS

### Definitions

In describing the present invention, the following terminology is used in accordance with the definitions set out below.

An “aquaculturally-raised” aquatic animal is one that was raised according to standard aquacultural practices, defined as the routine cultivation of finfish, crustaceans, or mollusks as described in the monographs “Intensive Fish Farming” (Shephard and Bromage 1992; McVey 1993), which are herein incorporated in their entireties.

An “Organic” certification for an aquatic animal requires that the animal is raised in such a way that 95% of the components in the feeds utilized for production are from certified Organic sources. The production processes used for these Organic fish, mollusks or crustaceans control the inputs and outputs of the production system to minimize the impact of aquatic animal production on the environment. For the purposes of this patent application capitalization is used to differentiate the statutory use of Organic as defined here from the chemical use of organic (i.e., a carbon containing compound) as outlined in the USDA National Organic Standards (2002).

A “100% Organic Seafood” is any aquatic animal raised in such a way that 100% of the feeds utilized for production are from certified Organic sources. The production process used for these Organic seafood controls the inputs and outputs of the production system to minimize the impact of seafood production on the environment. For the purposes of this patent application capitalization is used to differentiate the statutory use of Organic as defined here from the chemical use of organic as outlined in the USDA National Organic Standards (2002).

A “Finishing Feed” is a feed that is provided to an animal prior to harvest and not during the full course of production. This can be preferably as short as 1 day but can be up to two months.

An “aquatic animal” is an animal, which lives primarily in an aquatic environment and would include fish, crustaceans, and mollusks. For the purposes of this invention, the term “aquatic animal” shall be further limited to those animals for which aquaculture methods and/or commercial production practices have been developed, and thereby excludes all non-cultured or wild aquatic species as of the filing date of this application.

A “fish” and the plural “fish” are defined in this invention as any Ostiechthyean or Chondrichthyean fish, such as, but not limited to, sharks, rays, sturgeon, eels, anchovy, herring, carp, smelt, salmon, trout, hakes, cod, rockfish, bass, drum, mackerel, tuna, butterfish, catfish, flounder, and seabream.

A “mollusk” and the plural “mollusks” are defined in this invention as any shellfish from the phylum Molluska including bivalves, gastropods, cephalopods, and chitons such as, but not limited to, mussels, clams, oysters, scallops, snails, conch, abalone, squid and cuttlefish.

A “crustacean” and the plural “crustaceans” are defined in this invention as any member of the Class Crustacea, such as, but not limited to, shrimp, lobsters, red claws, and crabs.

### Embodiments

In one preferred embodiment, the present invention provides a catfish, or other fish, mollusks, or crustaceans, which has been selectively enriched with certain beneficial compounds, such as but not limited to, LC-PUFAs (e.g., DHA, ARA, EPA, etc.), carotenoids (e.g., lutein, β-carotene, astaxanthin, zeaxanthin, γ-carotene, lycopene, etc.), amino acids (e.g., taurine, arginine, methionine, lysine, cysteine, etc.) vitamins (e.g., vitamin A, vitamin C, vitamin E, etc.), minerals (e.g., iron, zinc, selenium, magnesium, etc.) or other beneficial compounds.

In another preferred embodiment, the present invention provides a feed composition which, when fed to fish, mollusks, or crustaceans, will selectively enrich the aquatic animal with certain health beneficial compounds, such as, but not limited to, LC-PUFAs (e.g., DHA, ARA, EPA, etc.), carotenoids (e.g., lutein, β-carotene, astaxanthin, zeaxanthin, γ-carotene, lycopene, etc.), amino acids (e.g., taurine, arginine, methionine, lysine, cysteine, etc.) vitamins (e.g., vitamin A, vitamin C, vitamin E, etc.), minerals (e.g., iron, zinc, selenium, magnesium, etc.) or other beneficial compounds.

In another preferred embodiment, the present invention provides a feed composition which is certified Organic and contains less than 5% animal material, or 100% Organic, and which when fed to fish, mollusks, or crustaceans, will selectively enrich the aquatic animal with certain health beneficial compounds, such as but not limited to, LC-PUFAs (e.g., DHA, ARA or arachidonic acid, EPA, etc.), carotenoids (e.g., lutein, β-carotene, astaxanthin, zeaxanthin, γ-carotene, lycopene, etc.), amino acids (e.g., taurine, arginine, methionine, lysine, cysteine, etc.); vitamins (e.g., vitamin A, vitamin C, vitamin E, etc.), minerals (e.g., iron, zinc, selenium, magnesium, etc.) or other beneficial compounds.

In another embodiment, the present invention provides a method to culture fish, mollusks, or crustaceans, selectively enriched with certain health beneficial compounds, such as, but not limited to, LC-PUFAs (e.g., DHA, ARA, EPA, etc.), carotenoids (e.g., lutein, β-carotene, astaxanthin, zeaxanthin, γ-carotene, lycopene, etc.), amino acids (e.g., taurine, arginine, methionine, lysine, cysteine, etc.) vitamins (e.g., vitamin A, vitamin C, vitamin E, etc.), minerals (e.g., iron, zinc, selenium, magnesium, etc.) or other beneficial compounds which allows the production of Organic or 100% Organic seafood products by using a feed that is Organic or 100% Organic. Included in this invention is the use of the Organic, or 100% Organic seafood product as a food for humans or feed for animals.

In yet another embodiment, the present invention provides a method for the use of these designer fish, mollusks or crustaceans, selectively enriched with certain health beneficial compounds, such as but not limited to, LC-PUFAs (e.g., DHA, ARA, EPA, etc.), carotenoids (e.g., lutein, β-carotene, astaxanthin, zeaxanthin, γ-carotene, lycopene, etc.), amino acids (e.g., taurine, arginine, methionine, lysine, cysteine, etc.) vitamins (e.g., vitamin A, vitamin C, vitamin E, etc.), minerals (e.g., iron, zinc, selenium, magnesium, etc.) or other beneficial compounds as food or feed.

Production Methods

Standard aquaculture practices have been well described (Shephard and Bromage 1992; McVey 1993), and completely contained aquaculture systems have also been described and are included herein by reference (Jory, McMahon et al. 2002). Such systems that are known in the art can be used for the practice of this invention. Alternatively, semi-intensive production systems are also well known in the art and can also be used (Lopez, Allen et al. 2002). These may include cages, pens, ponds, tanks, and any other open or closed production system. One key component to this invention is the unexpected result of the selective enrichment of the aquacultured animal products following feeding of the aquacultured animal with feeds as described herein containing beneficial compounds, such as LC-PUFAs, essential amino acids, pigments, and etc.

Organic Production Methods

Controlled aquatic production systems for finfish, shellfish and crustaceans, are known to those familiar with the art (Jory, McMahon et al. 2002). Organic management practices are defmed by U.S. and international regulation (e.g., USDA National Organic Standards 2002) and can be followed for the production of “Organic” seafood (Hardy 2002). Full certification, however, requires that the feed components not be of animal origin. To date, the complete elimination of fish byproducts (meal and oil) from the feeds of aquatic animals has not been accomplished (Hardy 2002). A solution to improve consumer acceptance of aquacultured fish, mollusks or crustaceans is the production of Organic seafood or 100% Organic seafood which are raised under controlled conditions and fed diets that consist either 95%, or entirely, of certified Organic ingredients (within the limits and qualifications set by the USDA). The present invention describes for the first time a totally vegetarian diet for fish, mollusks, and crustaceans, wherein the fishmeal and/or fish oil is replaced by a combination of hydrolyzed plant protein, bacteria, and microalgae containing omega-3 LC-PUFAs. Such diets will support growth of marine animals in the absence of fishmeal/oil. With the selection of dietary components that are themselves “Organic,” as defined by standard Organic certifying bodies such as the National Organic Standards Board (NOSB) or the like, these novel feeds could also be classified for the first time as “Organic Feeds.” Such feeds would include only non-genetically modified feed materials, no antibiotics, and no fishmeal or fish oil. The feeding of fish, mollusks or crustaceans using management practices known in the art that would also be considered “Organic” by the standard Organic certifying bodies such as NOSB, would result in an animal that would have a unique composition and be classified for the first time as, for example, an “Organic Shrimp,” “Organic Catfish,” or “Organic Clam” under the criteria of standardized Organic certification bodies, such as NOSB. Production of 100% Organic seafood will require that all inputs be Organic certified and production methods approved through the NOSB. This invention also encompasses the use of these Organic feeds for terrestrial animals such as, but not limited to, pigs, cows, chickens, and companion animals (e.g., cats, dogs, horses, etc.)

Pigment Enrichment

This invention also embodies the production of fish, mollusks, or crustaceans with a high level of lutein and/or zeaxanthin. The main carotenoid of wild type or cultured fish and shrimp is astaxanthin (Meyers and Latscha 1997). Chemically synthesized canthaxanthin has been used in some cases as a substitute for astaxanthin in salmon feeds, but its use has been recently limited due to concerns over the accumulation of canthaxanthin crystals in the retina of the eye (Goralczyk, Barker et al. 2000). There are no reports of shrimp or salmonid fish where astaxanthin was not the predominant carotenoid of the animal. The inventors surprisingly found that modulation of the dietary carotenoids could result in an aquatic animal where the main carotenoid was not astaxanthin. That is, where astaxanthin comprises less than 50% of the total carotenoid. An alternative result would be a significant modulation of the carotenoid profile where carotenoids not naturally found to accumulate to greater than 5% of the total carotenoid fraction have been enriched beyond 10% by alteration of diet. Dietary supplementation of the shrimp with certain materials including, but not limited to, marigold petals, Lycium Chinese Mill Berries, tomato products, maize gluten, certain microalgae, such as, but not limited to Chlorella, Spirulina, Crypthecodinium, Schizochytrium, diatoms, certain bacteria, such as, but not limited to Flavobacterium, and/or extracts from any of these sources, can be used to elevate the levels of lutein, zeaxanthin, lycopene and other carotenoids in the tissues of the animal. Surprisingly, these sources do not result in the complete conversion of the added carotenoids into astaxanthin.

The invention also envisions the use of artificial pigments including, but not limited to, lutein, zeaxanthin, lycopene, γ-carotene, and β-carotene, but the inclusion of these materials would not result in a 100% Organic certification of the feed or the aquatic animal consuming said feed. In both cases, however, a seafood product will be produced which contains less than 50-75% the total carotenoids as astaxanthin. Lutein, zeaxanthin or lycopene in their various forms are added to the standard feed to provide final carotenoid concentrations from 1 mg to 10 g per kg feed. Alternative carotenoids that are known to be present in crustaceans and are chosen from the following group, doradexanthin, idoxanthin, tetrol, α-cryptoxanthin, β-cryptoxanthin, echineone, 4-hydroxy-echineone, canthaxanthin, β-apo-8′-carotenal, phoenicoxanthin, isocryptoxanthin, adonixanthin (Meyers and Latscha 1997) can be added to the diet, via various algal strains or synthetic methods. These will enhance the visual profile of the cultured animals.

This invention also encompasses the delivery of the carotenoid in association with a phospholipid to make it more bioavailable. Vegetarian sources of astaxanthin are known in the art and can be supplied from the alga Hematococcus (Lorenz and Cysewski 2000) or the yeast Phaffia (Ramirez, Gutierrez et al. 2001). Using specific organic production methods described in this patent, these sources could be used as Organic sources of astaxanthin. These sources, as well as other sources of lutein, zeaxanthin and lycopene as described above, can be used in combination with phospholipids to significantly improve the bioavailability of the carotenoid in fish, mollusks or crustaceans. Phospholipids that are plant based can be used (e.g., soy lecithin), but phospholipids rich in LC-PUFAs (e.g., DHA rich phospholipids extracted from marine algae, egg phospholipids, fish extracts, etc.) are a preferred embodiment of this invention. The most preferred embodiment would be the phospholipids extracted from marine algae, as this would represent an Organic feed supplement.

Fatty Acid Profile Enhancement

Shrimp are known to contain a small amount of DHA (144 mg/100 g cooked shrimp) and typically have a DHA/EPA ratio of 0.8 (USDA 2002). Catfish also have only a small amount of DHA (128 mg DHA/100 g cooked catfish), whereas wild tuna, is very rich in DHA, containing about 1,141 mg DHA/100 g cooked tuna) with a DHA/EPA ratio of 3.1 (USDA 2002). Another source of DHA levels in foods can be found in the publication by Simopoulos and Robinson and are incorporated herein by reference (Simopoulos and Robinson 1998 ). High levels of DHA, such as in tuna, are considered healthful. DHA has many specific health benefits and it would be beneficial to elevate the DHA level of aquatic animals, which are naturally relatively low in DHA, such as shrimp, catfish, or tilapia. However, the use of fish oil or fishmeal to elevate DHA levels in these animals would result in a product that would no longer be certifiable as Organic by many Organic certifying bodies. Furthermore, the elevation of DHA via fish oil would be accompanied by an unwanted elevation of the EPA level. Elevated EPA levels are associated with reduced growth and increased bleeding times in humans and would therefore not be a beneficial attribute to the seafood product. Feeding of a DHA source including, but not limited to, certain microalgae (e.g., Crypthecodinium, Schizochytrium, etc.) or the extracts therefrom, particularly a phospholipid extract as described in U.S. Pat. No. 6,372,460, effectively elevates the DHA levels of the fish, mollusk, or crustacean relative to the levels found in wild species or aquaculturally-raised species. Furthermore, the DHA/EPA levels can be increased relative to that of the wild species or aquaculturally-raised species. The source of DHA is added at a level that provides DHA content in the feed from 1% of the total fat in the feed to 50% of the total fat in the feed. Since the microalgal biomass containing DHA as well as biomass from other algae or fungi containing ARA, EPA, and other LC-PUFAs can be grown in defined conditions using non-GMO strains, these materials can be certified Organic, and their production methods can be certified Organic. Consequently, this approach for the first time allows one to petition for Organic certification for any animal produced in such a defined system.

In some embodiments, the supplementation is sufficient to provide lutein at a level of at least about 60 mg/kg lutein, from about 60 mg/kg to about 200 mg/kg lutein, from about 200 mg/kg to about 500 mg/kg lutein, or from about 60 mg/kg to about 500 mg/kg lutein. In some embodiments, the supplementation is sufficient to provide zeaxanthin at a level of at least about 60 mg/kg zeaxanthin, from about 60 mg/kg to about 200 mg/kg zeaxanthin, from about 200 mg/kg to about 500 mg/kg zeaxanthin, or from about 60 mg/kg to about 500 mg/kg zeaxanthin. In some embodiments, the supplementation is sufficient to provide DHA at a level of at least about 12 mg/kg DHA, from about 12 mg/kg to about 24 mg/kg DHA, from about 24 mg/kg to about 40 mg/kg DHA, or from about 12 mg/kg to about 40 mg/kg DHA.

Supplementation of LC-PUFAs in a diet should also be accompanied by additional natural antioxidants such as, but not limited to, tocopherol and derivatives (e.g., Vitamin E), ascorbic acid and derivatives (e.g., Vitamin C), selenium, organoselenium compounds (e.g., garlic extract), carotenoids, or chemical antioxidants (e.g., butylated hydroxytoluene, benzoquinones, etc.). The levels of addition of such antioxidants are well known in the art. For example, Muggli provides a formula for calculating the amount of Vitamin E to be added to a product based on the amount of LC-PUFA in that product (Muggli 1989).

Taurine Enhancement

Fish and terrestrial animals are considered good sources of taurine. However, certain fish, mollusks, and crustaceans that have been raised using modern husbandry techniques can be relatively low in taurine (Takeuchi 2001). This is particularly the case for animals that are generally herbivorous (e.g., catfish, tilapia, carp, etc.). Supplementation of aquatic feeds with fishmeal allows for some enrichment of taurine in the feed. However, this would disallow an Organic certification, as the feeds would contain animal products and byproducts. Diets can be prepared with supplemental taurine produced by a fermentation process from yeast, bacteria, algae, or fungi naturally selected to overexpress this amino acid. Such diets would be considered vegetarian and could, therefore, qualify as Organic if appropriate care was taken in the fermentation process. Yeast, bacteria, fungi or algae can be used directly or an extract thereof can be used as the feed additive. The taurine containing material (0.01 to 100 g taurine per kg feed) is added to the feed at sufficient quantity to increase the taurine content of the aquatic animal by over 50% compared to animals that have been raised on diets containing no fishmeal, or over 25% compared to animals that have been raised on diets containing fishmeal. In some embodiments, the supplementation is sufficient to provide taurine at a level at least about 200 mg/kg taurine, from about 200 mg to about 1 g/kg taurine, from about 1 g/kg to about 2 g/kg taurine, or from about 200 mg to about 2 g/kg taurine.

Finishing Feed

Finishing feeds or additives containing any of the enrichments described above can be provided throughout the culture of the animal. Alternatively, and preferably, finishing feeds or additives are provided from 1 to 70 days prior to harvest to provide the final enrichment and change in composition of the animals in said beneficial component. Fish, mollusks, or crustaceans considered Organic or 100% Organic will need to be fed throughout the entire culture with the algal meal or extract and/or vegetable protein as a complete replacement for the fishmeal or fish oil. Other enrichments can be limited to the final three weeks of the cultivation cycle.

The following examples are provided for exemplification and are not intended to limit the scope of the invention.

### EXAMPLES

### Example 1

### Production of Catfish Containing High Levels of DHA

A minimum-water discharge pond is established for the intensive cultivation of catfish according to standard aquaculture practices (Sargent and Tacon 1999). Four weeks prior to harvest date, the feeding regimen of the catfish is altered to provide a Finishing Feed which comprises the standard catfish grow-out feed plus a supplement of algal DHA (25 g DHA/kg feed) provided as 200 g AQUAGROW ADVANTAGE/kg feed (Advanced BioNutrition Corp., Columbia, Md.). Alternatively, 300 g ALGAMAC 30/50/kg of feed can be used (Aquafauna BioMarine, Hawthorne, Calif.). The high-DHA catfish are harvested using processes and practices known in the art.

### Example 2

### Production of Tilapia Containing High Levels of DHA

Standard aquaculture practices with intensive, self-contained semi-intensive, or extensive tilapia production systems can be used. Intensive, minimum-water discharge systems would be preferable to produce Organic or 100% Organic high-DHA tilapia. Two weeks prior to harvest date, the feeding regimen of the tilapia is altered to provide a Finishing Feed which comprises the standard tilapia grow-out feed plus a supplement of algal DHA (12 g DHA/kg feed) provided as 100 g AQUAGROW ADVANTAGE/kg feed (Advanced BioNutrition Corp., Columbia, Md.). Alternatively, 150 g ALGAMAC 30/50/kg of feed can be used (Aquafauna BioMarine, Hawthorne, Calif.). The high-DHA tilapia are harvested using processes and practices known in the art.

### Example 3

### Production of Rainbow Trout Containing Lutein

Standard aquaculture practices with intensive, self-contained semi-intensive, or extensive rainbow trout production systems can be used. Intensive, minimum-water exchange systems would be preferable to produce an Organic high-lutein rainbow trout. Two weeks prior to harvest date, the feeding regimen of the trout is altered to provide a Finishing Feed which comprises the standard trout grow-out feed plus a supplement of lutein (60 mg lutein/kg feed) provided as a standardized marigold extract (6 mg lutein/100 mg oil) by the addition of 1 g marigold extract per kg feed. The high-lutein trout are harvested using processes and practices known in the art.

### Example 4

Production of Rainbow Trout Containing Astaxanthin and DHA

Standard aquaculture practices with intensive and self-contained, semi-intensive, or extensive rainbow trout production systems can be used. Intensive, zero-water exchange systems would be preferable to produce an Organic high-lutein rainbow trout. Two weeks prior to harvest date, the feeding regimen of the trout is altered to provide a Finishing Feed, which comprises the standard trout grow-out feed plus a supplement of astaxanthin provided by a mixture of Phaffia yeast (Igene Inc., Columbia, Md.) and AQUAGROW DHA (Advanced BioNutrition Corp, Columbia, Md.). Preferred proportions of Phaffia yeast to AQUAGROW DHA would be 1:1 to 1:10 and the mixture is added to the feed to provide a final astaxanthin level of from 1-10,000 ppm. The high-astaxanthin trout are harvested using processes and practices known in the art.

### Example 5

### Production of a High-Lutein/High-DHA Striped Bass

Standard aquaculture practices with intensive, self-contained semi-intensive, or extensive striped bass production systems can be used. Intensive, minimum-water exchange systems would be preferable to produce an Organic high-lutein/high-DHA striped bass. One week prior to harvest date, the feeding regimen of the striped bass is altered to provide a Finishing Feed which comprises the standard grow-out feed plus a supplement of lutein (60 mg lutein/kg feed) provided by the addition of 3 g marigold petal meal (ca. 2% lutein by weight) per kg feed, and algal DHA (5,000 mg DHA/kg feed) provided by the addition of 40 g AQUAGROW DHA (Advanced BioNutrition Corp, Columbia, Md.). The high-lutein, high-DHA striped bass are harvested using processes and practices known in the art.

### Example 6

### Production of a High-Zeaxanthin/High-DHA Shrimp

Standard aquaculture practices with intensive, self-contained semi-intensive, or extensive shrimp production systems can be used (Leung and Moss 2000; Haws and Boyd 2001). Intensive, minimum-water exchange systems as described in U.S. Pat. No. 6,327,996 would be preferable to produce an Organic high-lutein/high-DHA shrimp. Two weeks prior to harvest date, the feeding regimen of the shrimp is altered to provide a Finishing Feed which comprises the standard grow-out feed plus a supplement of zeaxanthin (60 mg zeaxanthin/kg feed) provided by the addition of 30 g Lycium Chinese Mill Berries (ca. 0.2% zeaxanthin) per kg feed, and algal DHA (5,000 mg DHA/kg feed) provided by the addition of 40 g AQUAGROW DHA (Advanced BioNutrifion Corp, Columbia, Md.). The high-zeaxanthin/high-DHA shrimp are harvested using processes and practices known in the art.

### Example 7

### Production of Salmon containing Lycopene and DHA

Standard aquaculture practices with intensive, self-contained semi-intensive, or extensive salmon production systems can be used. Intensive, minimum-water exchange systems would be preferable to produce an Organic high-lycopene salmon. Two weeks prior to harvest date, the feeding regimen of the salmon is altered to provide a Finishing Feed, which comprises the standard trout grow-out feed plus a supplement of lycopene provided by a mixture of tomato extract (1 g lycopene/kg feed) and AQUAGROW DHA. Preferred proportions of lycopene to AQUAGROW DHA would be 1:1 to 1:10 and the mixture is added to the feed to provide a final lycopene level of 1%. The high-lycopene salmon are harvested using processes and practices known in the art.

### Example 8

### Production of a High-Lutein/High-DHA/High Taurine Shrimp

Standard aquaculture practices with intensive, self-contained semi-intensive, or extensive shrimp production systems can be used (Leung and Moss 2000; Haws and Boyd 2001). Intensive, minimum-water exchange systems as described in U.S. Pat. No. 6,327,996 would be preferable to produce an Organic high-lutein/high-DHA shrimp. Two weeks prior to harvest date, the feeding regimen of the shrimp is altered to provide a Finishing Feed which comprises the standard grow-out feed plus a supplement of lutein (60 mg lutein/kg feed) provided by the addition of 3 g marigold petals per kg feed, algal DHA (5,000 mg DHA/kg feed) provided by the addition of 40 g AQUAGROW DHA, and taurine (2 g/kg feed) provided by the addition of 2 g of a purified powder supplement made from yeast (Ajinomoto Corporation, Japan). The high-lutein/high-DHA/high taurine shrimp are harvested using processes and practices known in the art.

### Example 9

### Production of Catfish Containing High Levels of Taurine

Standard aquaculture practices with intensive, self-contained semi-intensive, or extensive catfish production systems can be used. Intensive, minimum-water discharge systems would be preferable to produce Organic or 100% Organic high-DHA catfish. Two weeks prior to harvest date, the feeding regimen of the catfish is altered to provide a Finishing Feed which comprises the standard catfish grow-out feed plus a supplement of taurine (2 g/kg feed) provided by the addition of 2 g of a purified powder supplement made from yeast (Ajinomoto Corporation, Japan). The high-taurine catfish are harvested using processes and practices known in the art.

### Example 10

### Production of 100% Organic Shrimp

An intensive, minimum-water exchange production system (Leung and Moss 2000) would be preferable to produce an Organic shrimp. Such a system would be managed under the guidelines of the NOSB as a fully Organic operation. The feed input to the system is totally vegetarian. Fishmeal is replaced on a protein-to-protein basis with SPC, a hydrolyzed non-GMO soy meal concentrate (ADM Corp, Decatur, Ill., USA) while fish oil is replaced on a DHA basis with the microalgal sources of DHA. Typical shrimp grow-out feed will contain about 7 g DHA/kg. Certified Organic feed is prepared using non-GMO soy meal at an amount equivalent to the fishmeal protein component of a standard feed and ALGAMAC 30/50 (an algal DHA source containing about 10% DHA by weight) at 70 g/kg of final finished feed.

This certified Organic feed contains no antibiotics or other preservative chemicals. The shrimp are fed the Organic feed, which is produced in small particulate form for small shrimp and larger standardized pellets for larger shrimp, using procedures standard in the industry. This Organic feed is used up to the time of harvest, unless an Organic Finishing Feed is utilized (as envisioned by the instant invention). The Organic shrimp are then harvested using processes and practices known in the art. These shrimp are distinguished biochemically by a high-DHA/EPA ratio. This ratio will be greater than 1:1.

### Example 11

### Production of Organic shrimp

As in Example 10 except that as much as 4.99% of the ingredients do not have to be certified as Organic.

### Example 12

### Production of High DHA and High Zeaxanthin Organic Shrimp

Shrimp produced as in Examples 10 and 11 will have an Organic certification but will only have a standard DHA level (ca. DHA is 10-12% of total lipid) and little or no detectable zeaxanthin. The DHA levels are improved to over 15% of the total lipid, and significant levels or zeaxanthin are incorporated into the shrimp when the DHA/zeaxanthin-enriched Finishing Feed and process described in Example 6 is used as a Finishing Feed for 14 days prior to the harvest.

### Example 13

### Production of Organic Catfish

An intensive, controlled-water exchange production system would be preferable to produce an “Organic” catfish. Such as system would be managed under the guidelines of the NOSB as a fully Organic operation. The feed input to the system is totally vegetarian. Fishmeal is replaced on a protein-to-protein basis with hydrolyzed non-GMO soy meal while fish oil is replaced on a DHA basis with the microalgal sources of DHA. Typical catfish grow-out feed will contain about 1 g DHA/kg. Certified Organic feed is prepared using non-GMO soy meal at an amount equivalent to the fishmeal protein component of a standard feed and ALGAMAC 30/50 (an algal DHA source containing about 10% DHA by weight) at 10 g/kg of final feed.

This certified Organic feed contains no antibiotics or other preservative chemicals. The catfish are fed the Organic feed, which is produced in small particulate form for small animals and larger standardized pellets for larger animals using procedures standard in the industry. This Organic feed is used up to the time of harvest, unless an Organic Finishing Feed is utilized (as envisioned by this invention). The Organic catfish are then harvested using processes and practices known in the art. These catfish are distinguished biochemically by a high-DHA/EPA ratio.

### Example 14

### Production of High-DHA Organic Catfish

Catfish produced as in Example 13 will have an Organic certification but will only have a standard DHA level (ca. DHA is 3% of total lipid). The DHA levels are improved to over 5% of the total lipid in the catfish when the DHA-enriched Finishing Feed and process described in Example 1 is used.

### Example 15

### Production of High-DHA Organic Salmon

Salmon are produced using the same Organic practices as described for catfish in Example 14 but with the substitution of salmon feed for catfish feed. These salmon will have an Organic certification but will only have a standard DHA level (ca. DHA is 10% of total lipid). The DHA levels are improved to over 15% of the total lipid in the salmon when the DHA-enriched Finishing Feed and process described in Example 1 is used.

### Example 16

### High-DHA and High-Taurine Feed for Cats

A feed is prepared for cats by starting with a commercial cat diet and adding a mixture containing DHA (5,000 mg DHA/kg feed) from microalgae provided by the addition of 40 g AquaGrow DHA (a commercial DHA product of Advanced BioNutrition Corp) and taurine (2,000 mg taurine/kg feed) provided by the addition of 2 g purified powdered supplement of taurine made from yeast (Ajinimoto Corp, Japan).

### Example 17

### High-DHA and High-Taurine Organic Feed for Pigs

An Organic feed is prepared for pigs by starting with a commercial pig diet and adding a mixture containing algal DHA (5,000 mg DHA/kg feed) from microalgae provided by the addition of 50 g ALGAMAC 30/50 and taurine (2,000 mg taurine/kg feed) provided by the addition of taurine containing yeast (Ajinimoto Corp, Japan).

### REFERENCES

Burke, P. A., P. R. Ling, et al. (2001). “Sites of conditional essential fatty acid deficiency in end stage liver disease.” JPEN J Parenter Enteral Nutr 25(4): 188-93.

Chesney, R. W., R. A. Helms, et al. (1998). “An updated view of the value of taurine in infant nutrition.” Adv Pediatr 45: 179-200.

Chetty, N., S. C. Reavis, et al. (1989). “Fatty acid composition of some South African fresh-water fish.” S Afr Med J 76(7): 368-70.

Crawford, M. A., M. Bloom, et al. (1999). “Evidence for the unique function of docosahexaenoic acid during the evolution of the modern hominid brain.” Lipids 34(47): S39-47.

Goralczyk, R., F. M. Barker, et al. (2000). “Dose dependency of canthaxanthin crystals in monkey retina and spatial distribution of its metabolites.” Invest Ophthalmol Vis Sci 41 (6): 1513-22.

Gormley, J. J. (1999). DHA. A Good Fat, Kensington Publishing Corp. Hardy, R. (2002). Organic Farmed Fish. Aquaculture Magazine: 60-63.

Haws, M. and C. Boyd (2001). Methods for improving shrimp farming in Central America. Managua, Central American University Press.

Jory, D., D. McMahon, et al. (2002). Inland Shrimp Culture with Zero Water Exchange Ponds. Aquaculture Magazine: 74-77.

Kyle, D. J. (1997). “Production and use of a single cell oil highly enriched in arachidonic acid.” Lipid Technology 9: 116-121.

Kyle, D. J. and L. M. Arterburn (1998). “Single cell oil sources of docosahexaenoic acid: clinical studies.” World Rev Nutr Diet 83: 116-31.

Leung, P. S. and S. M. Moss (2000). Economic Assessment of a Prototype Biosecure Shrimp Growout Facility. Controlled and Biosecure Production Systems: Evolution and Integration of Shrimp and Chicken Production Models., Sidney, Australia, World Aquaculture Society.

Lombardini, J. B. (1992). “Review: recent studies on taurine in the central nervous system.” Adv Exp Med Biol 315: 245-51.

Lopez, M., R. Allen, et al. (2002). Projected Cost Comparison of Semi-Intensive, Zero-Exchange Culture Systems in Nicaragua. Global Aquaculture Advocate: 88-89.

Lorenz, R. T. and G. R. Cysewski (2000). “Commercial potential for Haematococcus microalgae as a natural source of astaxanthin.” Trends Biotechnol 18(4): 160-7.

Lourenco, R. and M. E. Camilo (2002). “Taurine: a conditionally essential amino acid in humans? An overview in health and disease.” Nutr Hosp 17(6): 262-70.

McVey, J. P. (1993). CRC Handbook of Mariculture: Crustacean Aguaculture. Boca Raton, Fla., CRC Press.

Meyers, S. P. and T. Latscha (1997). Carotenoids. Crustacean Nutrition, Baton Rouge, World Aquaculture Society.

Muggli, R. (1989). Dietary fish oils increase the requirement for Vitamin E in humans. St. John's, Newfoundland, ARTS Biomedical Publishers & Distributors.

Neuringer, M., H. Imaki, et al. (1987). “Abnormal visual acuity and retinal morphology in rhesus monkeys fed a taurine-free diet during the first three postnatal months.” Adv Exp Med Biol 217: 125-34.

Pita, M. L., J. M. Rubio, et al. (1997). “Chronic alcoholism decreases polyunsaturated fatty acid levels in human plasma, erythrocytes, and platelets—influence of chronic liver disease.” Thromb Haemost 78(2): 808-12.

Ramirez, J., H. Gutierrez, et al. (2001). “Optimization of astaxanthin production by Phaffia rhodozyrna through factorial design and response surface methodology.” J Biotechnol 88(3): 259-68.

Rapp, L. M., S. S. Maple, et al. (2000). “Lutein and zeaxanthin concentrations in rod outer segment membranes from perifoveal and peripheral human retina.” Invest Ophthalmol Vis Sci 41(5): 1200-9.

Salem, N., Jr. and R. J. Pawlosky (1994). “Arachidonate and docosahexaenoate biosynthesis in various species and compartments in vivo.” World Rev Nutr Diet 75: 114-9.

Sargent, J. R. and A. G. Tacon (1999). “Development of farmed fish: a nutritionally necessary alternative to meat.” Proc Nutr Soc 58(2): 377-83.

Shephard, C. J. and N. R. Bromage (1992). Intensive Fish Farming. Oxford, UK, Blackwell Scientific Publications.

Simopoulos, A. P. and J. P. Robinson (1998). The omega plan. New York, N.Y., Harper Collins.

Snodderly, D. M. (1995). “Evidence for protection against age-related macular degeneration by carotenoids and antioxidant vitamins.” Am J Clin Nutr 62(6 Suppl): 1448S-1461S.

Suleiman, M. S., A. C. Moffatt, et al. (1997). “Effect of ischaemia and reperfusion on the intracellular concentration of taurine and glutamine in the hearts of patients undergoing coronary artery surgery.” Biochim Biophys Acta 1324(2): 223-31.

Takeuchi, T. (2001). “Taurine content of Japanese flounder, Paralichthys olivaceus T. & S. and red sea bream Pagrus major T. & S. during the period of seed production.” Aguaculture Research 32: 244-248.

Tricarico, D., M. Barbieri, et al. (2001). “Voltage-dependent antagonist/agonist actions of taurine on Ca(2+)-activated potassium channels of rat skeletal muscle fibers.” J Pharmacol Exp Ther 298(3): 1167-71.

USDA (2002). USDA National Nutrient Data Base, USDA.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

1-80. (canceled)

81. An aquaculturally-raised aquatic animal made by cultivating an aquatic animal with a feed, wherein at least 1% by weight of the fat content of the feed is omega-3 long chain polyunsaturated fatty acid (O3-LC-PUFA) derived from a non-animal source.

82. The animal of claim 81, wherein the O3-LC-PUFA comprises at least one of docosahexaenoic acid (DHA), eicosapentaenoic acid (EPA), and arachidonic acid (ARA).

83. The animal of claim 81, wherein the O3-LC-PUFA comprises DHA.

84. The animal of claim 81, wherein from 1% to 50% by weight of the fat content of the feed is DHA.

85. The animal of claim 81, wherein the feed comprises less than 5% animal material.

86. The animal of claim 81, wherein the feed is substantially free of animal material.

87. The animal of claim 81, wherein the feed meets the organic certification requirements of the 2002 United States Department of Agriculture National Organic Program Standards (USDA NOPS).

88. The animal of claim 81, wherein the animal meets the organic certification requirements of the 2002 USDA NOPS.

89. The animal of claim 81, wherein the O3-LC-PUFA is derived from microalgae.

90. The animal of claim 89, wherein the microalgae are selected from the group consisting of Crypthecodinium, Schizochytrium, Thraustochytrium, Pavlova, Tetraselmis, and Isochrysis species, and combinations of these.

91. The animal of claim 81, comprising at least 12 grams of DHA per kilogram of animal.

92. The animal of claim 81, being a crustacean.

93. The animal of claim 81, being a shrimp.

94. The shrimp of claim 93, comprising more than 3 milligrams DHA per gram dry weight of shrimp.

95. The animal of claim 81, being a fish.

96. The fish of claim 95, wherein more than about 5% of the total extractable fat of the fish is DHA.

97. The animal of claim 81, being a catfish.

98. The animal of claim 81, being a mollusk.

99. The mollusk of claim 98, wherein more than about 10% of the total extractable fat of the mollusk is DHA.

100. The mollusk of claim 98, being an abalone.

101. The animal of claim 81, wherein the feed comprises at least one carotenoid derived from a non-animal source.

102. The animal of claim 101, wherein the carotenoid is selected from the group consisting of lutein, zeaxanthin, and lycopene.

103. The animal of claim 102, comprising at least about 60 milligrams lutein per kilogram of animal.

104. The animal of claim 102, comprising at least about 60 milligrams zeaxanthin per kilogram of animal.

105. The animal of claim 101, wherein less than about 50% of the total carotenoid content of the animal is astaxanthin.

106. The animal of claim 101, wherein the source is selected from the group consisting of marigold petals, Lycium Chinese Mill Berries, tomatoes, microalgae, diatoms, and bacteria.

107. The animal of claim 101, wherein the feed further comprises a phospholipid in an amount sufficient to enhance the bioavailability of the carotenoid.

108. The animal of claim 107, wherein the phospholipid is a phospholipid extracted from a marine algae.

109. The animal of claim 81, wherein the feed further comprises a taurine-containing material.

110. The animal of claim 109, wherein the feed comprises at least 10 milligrams taurine per kilogram.

111. The animal of claim 81, wherein the animal has been cultivated with the feed for at least two weeks prior to harvest.

112. A method of aquaculturally-raising an aquatic animal, the method comprising cultivating the animal with a feed, wherein at least 1% by weight of the fat content of the feed is omega-3 long chain polyunsaturated fatty acid (O3-LC-PUFA) derived from a non-animal source.

113. In a method of aquaculturally-raising an aquatic animal, the improvement comprising cultivating the animal with a feed, wherein at least 1% by weight of the fat content of the feed is omega-3 long chain polyunsaturated fatty acid (O3-LC-PUFA) derived from a non-animal source.

114. A feed for cultivating aquaculturally-raised aquatic animals, the feed comprising a fat component, wherein at least 1% by weight of the fat content of the feed is omega-3 long chain polyunsaturated fatty acid (O3-LC-PUFA) derived from a non-animal source.
